(19) United States
(12) Patent Application Publication
(10) Pub. No.: US 2006/0201601 A1
(43) Pub. Date: Sep. 14, 2006

(54) Flexible markers

(75) Inventors: Joseph G. Furst, Lyndhurst, OH (US); William Brodbeck, South Euclid, OH (US); Michael J. Wiggins, Cleveland, OH (US)

Correspondence Address: Fay, Sharpe, Fagan,;Minnich & McKee, LLP, 7th Floor, 1100 Superior Avenue, Cleveland, OH, 44114-2579, US

(73) Assignee: ICON Interventional Systems, Inc.

(21) Appl. No.: 11/325,476

(22) Filed: Jan. 4, 2006

Related U.S. Application Data
(60) Provisional application No. 60/658,412, filed on Mar. 3, 2005.

(51) Int. Cl. B31B 1/60; A61B 6/00

(52) U.S. Cl. 156/60; 600/431

(57) ABSTRACT

A flexible marker material that is visible to at least one form of electromagnetic wave, sound wave and/or magnetic wave. The marker material is used on a medical device and adds little or no bulk to the medical device.

The present invention claims priority on U.S. Provisional Patent Application Ser. No. 60/658,412 filed Mar. 3, 2005 entitled “Flexible Marker”, which is incorporated herein by reference.

The present invention is directed to medical devices, and particularly to medical devices that are used in various body passageways of humans and/or animals, and more particularly to a material that facilitates in visualizing a medical device during and/or after a medical procedure involving use of the medical device.

### BACKGROUND OF THE INVENTION

Various types of vascular medical devices currently include one or more markers to enable a surgeon to properly position or place a medical device the patient such as in a blood vessel during interventional cardiology. These medical devices that are used in interventional cardiology typically include balloon catheters, sheaths, stent catheters, electrophysiology catheters and the like. The markers used on the medical devices are typically visible under x-ray guidance. The markers are positioned on the medical device to enable the surgeon to correctly place the medical device in a patient during a particular medical procedure. During an interventional cardiology procedure, hoops of marker material are commonly positioned about an end portion of a hypotube. An angioplasty balloon is then welded or otherwise secured to the end portion of the hypotube. The marker bands on the hypotube are then used to indicate the position of the angioplasty balloon in a body passageway. If a stent is used, the stent is crimped to the angioplasty balloon. The markers on the hypotube are then used to indicate the position of the stent in a body passageway. The markers used on the hypotube typically consist of bulky, inflexible bands of metals. Typical metal materials used as markers are titanium, gold and tungsten. The bulk and inflexibility of these marker bands can interfere or prevent a medical device (e.g., combination hyptotube and angioplasty balloon; combination hyptotube, angioplasty balloon and stent, etc.) from being properly positioned in a desired region of a blood vessel. As such, the medical device cannot effectively reach the site of treatment, or requires increased time and effort for completion of a successful treatment. For instance, during an angioplasty procedure, a blockage in a blood vessel is identified and then diagnosed for treatment. Typically, a balloon catheter or a stent is positioned at the site of the blockage for treatment. At times this blockage is very hard and/or contains calcium and/or may be in a very tortuous area where greater flexibility is needed for procedural success. The current marker bands on the catheters and hypotubes can interfere or prevent the medical devices from reaching the treatment areas due to the loss flexibility from the bands and/or due to the bulkiness of the bands. As such, the medical device cannot enter or easily enter a treatment area.

A biodegradable marker material is disclosed in U.S. Pat. No. 6,174,330 and U.S. Pat. No. 6,626,936, both of which are incorporated herein by reference. This biodegradable marker material is designed degrade and enter into the body of a patient over a period of time. A marker material is then coated on a stent and a protective coating material is applied to the marker material to inhibit corrosion is disclosed in U.S. Pat. No. 6,174,329, which is incorporated herein by reference. A marker material in the form of fibers that is included in a woven fiber material for a vasooccusive device is disclosed in U.S. Pat. No. 5,423,849, which is incorporated herein by reference.

In view of the current state of the art with respect to medical devices, there is a need for a medical device that includes one or more markers that is flexible and adds little or no weight or thickness to the medical device.

### SUMMARY OF THE INVENTION

The present invention is directed to a marker material that is a flexible material that adds little or no weight and/or thickness (i.e. bulkiness, etc.) to the medical device. As such, the marker material of the present invention enables a medical device to be more easily and is better able to be properly positioned in a treatment area such as a body passageway. As defined herein, the term “body passageway” is defined to be any passageway or cavity in a living organism (e.g., bile duct, bronchiole tubes, nasal cavity, blood vessels, heart, esophagus, trachea, stomach, fallopian tube, uterus, ureter, urethra, the intestines, lymphatic vessels, nasal passageways, eustachian tube, acoustic meatus, etc.). The techniques employed to deliver the medical device to a treatment area include, but are not limited to, angioplasty, vascular anastomoses, transplantation, implantation, subcutaneous introduction, minimally invasive surgical procedures, injection, topical applications, bolus administration, infusion, interventional procedures, and any combinations thereof. For vascular applications, the term “body passageway” primarily refers to blood vessels and chambers in the heart. When the medical device is in the form of a stent, the stent can be an expandable stent that is expandable by a balloon and/or other means. The stent can have many shapes and forms. Such shapes can include, but are not limited to, stents disclosed in U.S. Pat. Nos. 6,206,916 and 6,436,133; and all the prior art cited in these patents. These various designs and configurations of stents in such patents are incorporated herein by reference. The marker material of the present invention is designed to be visible to electromagnetic waves (e.g., x-rays, micro-waves, visible light, infrared waves, ultraviolet waves, etc.); sound waves (e.g, ultrasound waves, etc.); magnetic waves (e.g., MRI, etc.); and/or other types of electromagnetic waves (e.g., micro-waves, visible light, infrared waves, ultraviolet waves, etc.). In one non-limiting embodiment, the marker material is visible to x-rays (i.e., radiopaque). The marker material of the present invention is also designed to be a flexible material and/or be used in a flexible arrangement so as to not adversely interfere or insignificantly interfere with the flexibility of the medical device. One or more regions of a medical device can be formed of and/or include the marker material. The marker material can also and/or alternatively be designed to be at least partially coated on a medical device so as to not add a significant amount of bulk to the medical device. The coated marker material can be applied on the complete surface of the medical device or be selectively applied to one or more regions on the medical device. The marker material includes at least three components, namely an adhesive material, a metal powder material and a protective material. As can be appreciated, the marker material can include additional components. The coating thickness of the marker material of the present invention is less than about 1000 microns. In one non-limiting arrangement, the coating thickness of the marker material of the present invention is less than about 800 microns. In another non-limiting arrangement, the coating thickness of the marker material of the present invention is less than about 600 microns. In still another non-limiting arrangement, the coating thickness of the marker material of the present invention is less than about 400. In yet another non-limiting arrangement, the coating thickness of the marker material of the present invention is less than about 300 microns. In still yet another non-limiting arrangement, the coating thickness of the marker material of the present invention is less than about 275 microns. In a further non-limiting arrangement, the coating thickness of the marker material of the present invention is about 10-250 microns. In still a further non-limiting arrangement, the coating thickness of the marker material of the present invention is about 15-200 microns. In yet a further non-limiting arrangement, the coating thickness of the marker material of the present invention is about 15-150 microns. As can be appreciated, other coating thicknesses can be used.

In one non-limiting aspect of the present invention, the metal powder has a particle size wherein at least about 95% of the metal powder particles has an average cross-sectional area that is less than about 200 microns (about 65-70 mesh). In one non-limiting arrangement, the average particle size of at least about 95% of the metal powder is less than about 175 microns (about 70-80 mesh). In another non-limiting arrangement, the average particle size of at least about 95% of the metal powder is less than about 150 microns (about 90-110 mesh). In still another non-limiting arrangement, the average particle size of at least about 95% of the metal powder is less than about 100 microns. In yet another non-limiting arrangement, the average particle size of at least about 95% of the metal powder is about 10-75 microns. In still yet another non-limiting arrangement, the average particle size of at least about 95% of the metal powder is about 15-60 microns. In a further non-limiting arrangement, the average particle size of at least about 99.9% of the metal powder is less than about 200 microns. In still a further-non-limiting arrangement, the average particle size of at least about 99.9% of the metal powder is less than about 150 microns. In yet a further non-limiting arrangement, the average particle size of at least about 99.9% of the metal powder is about 10-75 microns. In still yet a further non-limiting arrangement, the average particle size of at least about 99.9% of the metal powder is about 15-60 microns. In another non-limiting arrangement, the average particle size of at least about 99.99% of the metal powder is about 10-75 microns. A non-limiting list of metal powders that can be used include, but are not limited to, aluminum, barium, bismuth, calcium, cobalt, copper, chromium, depleted radioactive elements, gold, holmium, iridium, iron, lead, molybdenum, nickel, niobium, osmium, palladium, platinum, rare earth metals, rhenium, rhodium, ruthenium, silver, stainless steel, tantalum, titanium, tungsten, vanadium, yttrium, zinc, zirconium, and/or an alloy that includes two or more of such metals. As can be appreciated, other and/or additional metals can be used. In another non-limiting arrangement, the metal powder includes a majority of bismuth, molybdenum, rhenium, tungsten and/or an alloy that include two or more of such metals. In another non-limiting arrangement, the metal powder includes a majority of bismuth. In still another non-limiting arrangement, the metal powder includes a majority of molybdenum. In yet another non-limiting arrangement, the metal powder includes a majority of rhenium. In still yet another non-limiting arrangement, the metal powder includes a majority of tungsten. The metal powder in the flexible marker material generally constitutes less than about 85 percent of the total thickness of the marker material so as to not adversely affect the flexibility of the marker material. In one non-limiting arrangement, the average thickness of the metal powder constitutes about 25-75 percent of the total thickness of the marker material. As can be appreciated, the average thickness of the metal powder can constitute other percentages of the total thickness of the marker material. The average weight percent of the metal powder in the marker material is generally less than about 98 percent of the marker material. In one non-limiting arrangement, the average weight percent of the metal powder in the marker material is about 30-95 percent of the marker material. In another non-limiting arrangement, the average weight percent of the metal powder in the marker material is about 50-95 percent of the marker material. In still another non-limiting arrangement, the average weight percent of the metal powder in the marker material is about 60-95 percent of the marker material. As can be appreciated, the average weight percent of the metal powder in the marker material can constitute other weight percentages. In still yet another non-limiting arrangement, the metal powder can be partially or fully substituted with a metal salt that has a density that is equal to or greater than the density of metal bismuth. The size parameters of the metal salt, when used, are similar to the size constraints of the metal powder as set forth above. The thickness of the metal powder and metal salt layer or only metal salt layer, when metal salt is used, is similar to the layer thickness constraints when using only metal powder as set forth above. The weight percent of the metal powder and metal salt layer or only metal salt layer, when metal salt is used, is similar to the weight percent constraints when using only metal powder as set forth above.

In another and/or alternative non-limiting aspect of the present invention, the marker material can include a variety of adhesives. The adhesive material is generally a medical grade adhesive; however, this is not required. The adhesive material can be a biostable or biodegradable material (i.e., dissolves, degrades, is absorbed, or any combination thereof in the body). The adhesive material is typically a flexible material. One non-limiting adhesive material that can be used includes a silicone material. Various types of adhesive materials can be used such as, but not limited to, a silicone or silicone based adhesive, a polyurethane or polyurethane based adhesive, and acrylic or acrylic based adhesive, etc. One non-limiting silicone material includes a silastic material. Non-limiting silastic material includes a silastic silicone rubber material. This material can include silica; however, this is not required. As can be appreciated, other or additional silicone materials can be used as the adhesive material. The adhesive material is at least partially formulated to adhere the metal powder to the medical device. The adhesive material can also or alternatively be formulated to adhere the protective material to the medical device. Typically, the adhesive material is applied to one or more portions of the medical device prior to applying the metal powder; however, this is not required. Indeed, the metal powder can be at least partially applied before and/or simultaneously with the adhesive material. At least a portion of the metal powder can also or alternatively be mixed with the adhesive material prior to applying the adhesive material to the medical device. When the adhesive material is at least partially applied to the medical device prior to all or a portion of the metal powder being applied to the medical device, at least a portion of the metal powder can be applied to the adhesive material on the medical device in a substantially pure form or be mixed with adhesive material and/or the protective material prior to being applied to the adhesive material on the medical device. The average thickness of the adhesive material coating on the medical device prior to any metal powder and/or protective material being applied to the adhesive material is generally less than about 100 microns. In one non-limiting arrangement, the average thickness of the adhesive material coating on the medical device prior to any metal powder and/or protective material being applied to the adhesive material is less than about 75 microns. In still another non-limiting arrangement, the average thickness of the adhesive material coating on the medical device prior to any metal powder and/or protective material being applied to the adhesive material is about 2-50 microns. In yet another non-limiting arrangement, the average thickness of the adhesive material coating on the medical device prior to any metal powder and/or protective material being applied to the adhesive material is about 3-20 microns. As can be appreciated, other average coating thicknesses of the adhesive material can be used. The adhesive material can be coated on the medical device by a variety of mechanisms such as, but not limited to, spraying (e.g., atomizing spray techniques, air-brushing, spraying, etc.), dip coating, roll coating, sonication, brushing, vapor deposition, etc. A masking technique can be used to limit the location of the adhesive on the medical device; however, this is not required. One or more of these techniques also or alternatively can be used to apply the metal powder and/or protective material to the medical device; however, this is not required. In one non-limiting arrangement, the adhesive material is a silastic medical grade adhesive material that is diluted with a solvent (e.g., silicone oil, etc.) and spray coated on one or more portions of the medical device. The viscosity of the diluted adhesive material can be about 0.2-3 cst; however, other viscosities can be used. In another one non-limiting arrangement, an adhesive material that includes octamethyltrisiloxane is used on the medical device. About one gram of this adhesive material is diluted with about 3 ml of silicone oil to form a mixture having a viscosity of about 0.8-1.3 cst. The adhesive material can be formulated to be rapidly set by use of heat, radiation, chemical reaction, light, etc.; however, this is not required.

In still another and/or alternative non-limiting one aspect of the present invention, the protective material is formulated to at least partially form a barrier between the metal powder and/or adhesive on the medical device and the external environment of the medical device when inserted in a treatment area. This barrier can function as a substantially permanent barrier or be a temporary barrier for the time period the medical device is in a treatment area. The protective material can also be used to facilitate in retaining the metal powder and/or adhesive on the medical device, 2) shield the metal powder layer and/or adhesive layer from damage during a medical procedure, and/or 3) provide a desired surface profile on the medical device. The protective material can be a biostable material or a biodegradable material (i.e., dissolves, degrades, is absorbed, or any combination thereof in the body). The protective material can a porous material or a non-porous material. The average thickness of the protective material coating on the medical device is generally less than about 50 microns. In one non-limiting arrangement, the average thickness of the protective material coating on the medical device is less than about 25 microns. In still another non-limiting arrangement, the average thickness of the protective material coating on the medical device is about 0.1-10 microns. In yet another non-limiting arrangement, the average thickness of the protective material coating on the medical device is about 0.3-5 microns. As can be appreciated, other average coating thicknesses of the protective material can be used. The protective material can be coated on the medical device by a variety of mechanisms such as, but not limited to, spraying (e.g., atomizing spray techniques, air-brushing, spraying, etc.), dip coating, roll coating, sonication, brushing, vapor deposition, etc. A masking technique can be used to limit the location of the protective material on the medical device; however, this is not required. In one embodiment of the invention, the protective material includes one or more sugars (e.g., glucose, fructose, sucrose, etc.), carbohydrate compounds, salts (e.g., NaCl, etc.), and/or polymers. In one aspect of this embodiment, the protective material includes one or more polymers. The one or more polymers that can be used can be biodegradable, bioresorbable, or bioerodable; polymers that are considered to be biostable; and/or polymers that can be made to be biodegradable and/or bioresorbable with modification. The protective material is typically a biostable material when the medical device (e.g., stent, PFO (patent foramen ovale) device, other types of grafts, prosthetic device, etc.) is designed to be left in a body passageway, or potentially will be in a body passageway for an extended period of time; however, it will be appreciated that a biodegradable protective material can be used. When the medical device (e.g., guide catheter, guide wire, angioplasty balloon, etc.) is not designed to be left in the body or will not be in the body for an extended period of time or is shielded from body fluids while in a body passageway, the protective material can be a biostable material or biodegradable material (i.e., dissolves, degrades, is absorbed, or any combination thereof in the body); however, this is not required. Non-limiting examples of polymers that are considered to be biodegradable, bioresorbable, or bioerodable include, but are not limited to, aliphatic polyesters; poly(glycolic acid) and/or copolymers thereof (e.g., poly(glycolide trimethylene carbonate); poly(caprolactone glycolide)); poly(lactic acid) and/or isomers thereof (e.g., poly-L(lactic acid) and/or poly-D Lactic acid) and/or copolymers thereof (e.g. DL-PLA), with and without additives (e.g. calcium phosphate glass), and/or other copolymers (e.g. poly(caprolactone lactide), poly(lactide glycolide), poly(lactic acid ethylene glycol)); poly(ethylene glycol); poly(ethylene glycol) diacrylate; poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-co-valerate); polyhydroxyalkaoates (PHA); polycaprolactone; poly(caprolactone-polyethylene glycol) copolymer; poly(valerolactone); polyanhydrides; poly(orthoesters) and/or blends with polyanhydrides; poly(anhydride-co-imide); polycarbonates (aliphatic); poly(hydroxyl-esters); polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarate-co-ethylene glycol); poly(fumarate anhydrides); fibrinogen; fibrin; gelatin; cellulose and/or cellulose derivatives and/or cellulosic polymers (e.g., cellulose acetate, cellulose acetate butyrate, cellulose butyrate, cellulose ethers, cellulose nitrate, cellulose propionate, cellophane); chitosan and/or chitosan derivatives (e.g., chitosan NOCC, chitosan NOOC-G); alginate; polysaccharides; starch; amylase; collagen; polycarboxylic acids; poly(ethyl ester-co-carboxylate carbonate) (and/or other tyrosine derived polycarbonates); poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof; poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; biodegradable polyesters; polydihydropyrans; polydepsipeptides; polyarylates (L-tyrosine-derived) and/or free acid polyarylates; polyamides (e.g., Nylon 66, polycaprolactam); poly(propylene fumarate-co-ethylene glycol) (e.g., fumarate anhydrides); hyaluronates; poly-p-dioxanone; polypeptides and proteins; polyphosphoester; polyphosphoester urethane; polysaccharides; pseudo-poly(amino acids); starch; terpolymer; (copolymers of glycolide, lactide, or dimethyltrimethylene carbonate); rayon; rayon triacetate; latex; and/pr copolymers, blends, and/or composites of above. Non-limiting examples of polymers that considered to be biostable include, but are not limited to, parylene; parylene c; parylene f; parylene n; parylene derivatives; maleic anyhydride polymers; phosphorylcholine; poly n-butyl methacrylate (PBMA); polyethylene-co-vinyl acetate (PEVA); PBMA/PEVA blend or copolymer; polytetrafluoroethene (Teflon®) and derivatives; poly-paraphenylene terephthalamide (Kevlar®); poly(ether ether ketone) (PEEK); poly(styrene-b-isobutylene-b-styrene) (Translute™); tetramethyldisiloxane (side chain or copolymer); polyimides polysulfides; poly(ethylene terephthalate); poly(methyl methacrylate); poly(ethylene-co-methyl methacrylate); styrene-ethylene/butylene-styrene block copolymers; ABS; SAN; acrylic polymers and/or copolymers (e.g., n-butyl-acrylate, n-butyl methacrylate, 2-ethylhexyl acrylate, lauryl-acrylate, 2-hydroxy-propyl acrylate, polyhydroxyethyl, methacrylate/methylmethacrylate copolymers); glycosaminoglycans; alkyd resins; elastin; polyether sulfones; epoxy resin; poly(oxymethylene); polyolefins; polymers of silicone; polymers of methane; polyisobutylene; ethylene-alphaolefin copolymers; polyethylene; polyacrylonitrile; fluorosilicones; poly(propylene oxide); polyvinyl aromatics (e.g. polystyrene); poly(vinyl ethers) (e.g. polyvinyl methyl ether); poly(vinyl ketones); poly(vinylidene halides) (e.g. polyvinylidene fluoride, polyvinylidene chloride); poly(vinylpyrolidone); poly(vinylpyrolidone)/vinyl acetate copolymer; polyvinylpridine prolastin or silk-elastin polymers (SELP); silicone; silicone rubber; polyurethanes (polycarbonate polyurethanes, silicone urethane polymer) (e.g., chronoflex varieties, bionate varieties); vinyl halide polymers and/or copolymers (e.g. polyvinyl chloride); polyacrylic acid; ethylene acrylic acid copolymer; ethylene vinyl acetate copolymer; polyvinyl alcohol; poly(hydroxyl alkylmethacrylate); Polyvinyl esters (e.g. polyvinyl acetate); and/or copolymers, blends, and/or composites of above. Non-limiting examples of polymers that can be made to be biodegradable and/or bioresorbable with modification include, but are not limited to, hyaluronic acid (hyanluron); polycarbonates; polyorthocarbonates; copolymers of vinyl monomers; polyacetals; biodegradable polyurethanes; polyacrylamide; polyisocyanates; polyamide; and/or copolymers, blends, and/or composites of above. As can be appreciated, other and/or additional polymers and/or derivatives of one or more of the above listed polymers can be used. In one non-limiting arrangement, the protective material includes parylene and/or a parylene derivative.

In yet another and/or alternative non-limiting aspect of the present invention, the marker material can include and/or be coated with one or more biological agents. The term “biological agent” includes, but is not limited to, a substance, drug or otherwise formulated and/or designed to prevent, inhibit and/or treat one or more biological problems, and/or to promote the healing in a treated area. Non-limiting examples of biological problems that can be addressed by one or more biological agents include, but are not limited to, viral, fungus and/or bacteria infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; and/or the like. Non-limiting examples of biological agents that can be used include, but are not limited to, 5-Fluorouracil and/or derivatives thereof; 5-Phenylmethimazole and/or derivatives thereof; ACE inhibitors and/or derivatives thereof; acenocoumarol and/or derivatives thereof; acyclovir and/or derivatives thereof; actilyse and/or derivatives thereof; adrenocorticotropic hormone and/or derivatives thereof; adriamycin and/or derivatives thereof; agents that modulate intracellular Ca₂₊ transport such as L-type (e.g., diltiazem, nifedipine, verapamil, etc.) or T-type Ca₂₊ channel blockers (e.g., amiloride, etc.); alpha-adrenergic blocking agents and/or derivatives thereof; alteplase and/or derivatives thereof; amino glycosides and/or derivatives thereof (e.g., gentamycin, tobramycin, etc.); angiopeptin and/or derivatives thereof; angiostatic steroid and/or derivatives thereof; angiotensin II receptor antagonists and/or derivatives thereof; anistreplase and/or derivatives thereof; antagonists of vascular epithelial growth factor and/or derivatives thereof; anti-biotics; anti-coagulant compounds and/or derivatives thereof; anti-fibrosis compounds and/or derivatives thereof; anti-fungal compounds and/or derivatives thereof; anti-inflammatory compounds and/or derivatives thereof; Anti-Invasive Factor and/or derivatives thereof; anti-metabolite compounds and/or derivatives thereof (e.g., staurosporin, trichothecenes, and modified diphtheria and ricin toxins, Pseudomonas exotoxin, etc.); anti-matrix compounds and/or derivatives thereof(e.g., colchicine, tamoxifen, etc.); anti-microbial agents and/or derivatives thereof; anti-migratory agents and/or derivatives thereof (e.g., caffeic acid derivatives, nilvadipine, etc.); anti-mitotic compounds and/or derivatives thereof; anti-neoplastic compounds and/or derivatives thereof; anti-oxidants and/or derivatives thereof; anti-platelet compounds and/or derivatives thereof; anti-proliferative and/or derivatives thereof; anti-thrombogenic agents and/or derivatives thereof; argatroban and/or derivatives thereof; ap-1 inhibitors and/or derivatives thereof (e.g., for tyrosine kinase, protein kinase C, myosin light chain kinase, Ca₂₊/calmodulin kinase II, casein kinase II, etc.); aspirin and/or derivatives thereof; azathioprine and/or derivatives thereof; β-Estradiol and/or derivatives thereof; β-1-anticollagenase and/or derivatives thereof; calcium channel blockers and/or derivatives thereof; calmodulin antagonists and/or derivatives thereof (e.g., H₇, etc.); CAPTOPRIL and/or derivatives thereof; cartilage-derived inhibitor and/or derivatives thereof; ChIMP-3 and/or derivatives thereof; cephalosporin and/or derivatives thereof (e.g., cefadroxil, cefazolin, cefaclor, etc.); chloroquine and/or derivatives thereof; chemotherapeutic compounds and/or derivatives thereof (e.g., 5-fluorouracil, vincristine, vinblastine, cisplatin, doxyrubicin, adriamycin, tamocifen, etc.); chymostatin and/or derivatives thereof; CILAZAPRIL and/or derivatives thereof; clopidigrel and/or derivatives thereof; clotrimazole and/or derivatives thereof; colchicine and/or derivatives thereof; cortisone and/or derivatives thereof; coumadin and/or derivatives thereof; curacin-A and/or derivatives thereof; cyclosporine and/or derivatives thereof; cytochalasin and/or derivatives thereof (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin O, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.); cytokines and/or derivatives thereof; desirudin and/or derivatives thereof; dexamethazone and/or derivatives thereof; dipyridamole and/or derivatives thereof; eminase and/or derivatives thereof; endothelin and/or derivatives thereof; endothelial growth factor and/or derivatives thereof; epidermal growth factor and/or derivatives thereof; epothilone and/or derivatives thereof; estramustine and/or derivatives thereof; estrogen and/or derivatives thereof; fenoprofen and/or derivatives thereof; fluorouracil and/or derivatives thereof; flucytosine and/or derivatives thereof; forskolin and/or derivatives thereof; ganciclovir and/or derivatives thereof; glucocorticoids and/or derivatives thereof (e.g., dexamethasone, betamethasone, etc.); glycoprotein IIb/IIIa platelet membrane receptor antibody and/or derivatives thereof; GM-CSF and/or derivatives thereof; griseofulvin and/or derivatives thereof; growth factors and/or derivatives thereof(e.g., VEGF; TGF; IGF; PDGF; FGF, etc.); growth hormone and/or derivatives thereof; heparin and/or derivatives thereof; hirudin and/or derivatives thereof; hyaluronate and/or derivatives thereof; hydrocortisone and/or derivatives thereof; ibuprofen and/or derivatives thereof; immunosuppressive agents and/or derivatives thereof (e.g., adrenocorticosteroids, cyclosporine, etc.); indomethacin and/or derivatives thereof; inhibitors of the sodium/calcium antiporter and/or derivatives thereof (e.g., amiloride, etc.); inhibitors of the IP₃ receptor and/or derivatives thereof; inhibitors of the sodium/hydrogen antiporter and/or derivatives thereof (e.g., amiloride and derivatives thereof, etc.); insulin and/or derivatives thereof; Interferon alpha 2 Macroglobulin and/or derivatives thereof; ketoconazole and/or derivatives thereof; Lepirudin and/or derivatives thereof; LISINOPRIL and/or derivatives thereof; LOVASTATIN and/or derivatives thereof; marevan and/or derivatives thereof; mefloquine and/or derivatives thereof; metalloproteinase inhibitors and/or derivatives thereof; methotrexate and/or derivatives thereof; metronidazole and/or derivatives thereof; miconazole and/or derivatives thereof; monoclonal antibodies and/or derivatives thereof; mutamycin and/or derivatives thereof; naproxen and/or derivatives thereof; nitric oxide and/or derivatives thereof; nitroprusside and/or derivatives thereof; nucleic acid analogues and/or derivatives thereof (e.g., peptide nucleic acids, etc.); nystatin and/or derivatives thereof; oligonucleotides and/or derivatives thereof; paclitaxel and/or derivatives thereof; penicillin and/or derivatives thereof; pentamidine isethionate and/or derivatives thereof; phenindione and/or derivatives thereof; phenylbutazone and/or derivatives thereof; phosphodiesterase inhibitors and/or derivatives thereof; Plasminogen Activator Inhibitor-l and/or derivatives thereof; Plasminogen Activator Inhibitor-2 and/or derivatives thereof; Platelet Factor 4 and/or derivatives thereof; platelet derived growth factor and/or derivatives thereof; plavix and/or derivatives thereof; POSTMI 75 and/or derivatives thereof; prednisone and/or derivatives thereof; prednisolone and/or derivatives thereof; probucol and/or derivatives thereof; progesterone and/or derivatives thereof; prostacyclin and/or derivatives thereof; prostaglandin inhibitors and/or derivatives thereof; protamine and/or derivatives thereof; protease and/or derivatives thereof; protein kinase inhibitors and/or derivatives thereof (e.g., staurosporin, etc.); quinine and/or derivatives thereof; radioactive agents and/or derivatives thereof (e.g., Cu-64, Ca-67, Cs-131, Ga-68, Zr-89, Ku-97, Tc-99m, Rh-105, Pd-103, Pd-109, In-111, I-123, I-125, I-131, Re-186, Re-188, Au-198, Au-199, Pb-203, At-211, Pb-212, Bi-212, H₃P³²O₄, etc.); rapamycin and/or derivatives thereof; receptor antagonists for histamine and/or derivatives thereof; refludan and/or derivatives thereof; retinoic acids and/or derivatives thereof; revasc and/or derivatives thereof; rifamycin and/or derivatives thereof; sense or anti-sense oligonucleotides and/or derivatives thereof (e.g., DNA, RNA, plasmid DNA, plasmid RNA, etc.); seramin and/or derivatives thereof; steroids; seramin and/or derivatives thereof; serotonin and/or derivatives thereof; serotonin blockers and/or derivatives thereof; streptokinase and/or derivatives thereof; sulfasalazine and/or derivatives thereof; sulfonamides and/or derivatives thereof (e.g., sulfamethoxazole, etc.); sulphated chitin derivatives; Sulphated Polysaccharide Peptidoglycan Complex and/or derivatives thereof; T_{H1} and/or derivatives thereof (e.g., Interleukins-2, -12, and -15, gamma interferon, etc.); thioprotese inhibitors and/or derivatives thereof; taxol and/or derivatives thereof (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.); ticlid and/or derivatives thereof; ticlopidine and/or derivatives thereof; tick anti-coagulant peptide and/or derivatives thereof; thioprotese inhibitors and/or derivatives thereof; thyroid hormone and/or derivatives thereof; Tissue Inhibitor of Metalloproteinase-1 and/or derivatives thereof; Tissue Inhibitor of Metalloproteinase-2 and/or derivatives thereof; tissue plasma activators; TNF and/or derivatives thereof, tocopherol and/or derivatives thereof; toxins and/or derivatives thereof; tranilast and/or derivatives thereof; transforming growth factors alpha and beta and/or derivatives thereof; trapidil and/or derivatives thereof; triazolopyrimidine and/or derivatives thereof; vapiprost and/or derivatives thereof; vinblastine and/or derivatives thereof; vincristine and/or derivatives thereof; zidovudine and/or derivatives thereof. As can be appreciated, the biological agent can include one or more derivatives of the above listed compounds and/or other compounds. In one non-limiting embodiment, the biological agent includes, but is not limited to, trapidil, trapidil derivatives, taxol, taxol derivatives (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.), cytochalasin, cytochalasin derivatives (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin O, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.), paclitaxel, paclitaxel derivatives, rapamycin, rapamycin derivatives, 5-Phenylmethimazole, 5-Phenylmethimazole derivatives, GM-CSF (granulo-cyte-macrophage colony-stimulating-factor), GM-CSF derivatives, or combinations thereof. The type and/or amount of biological agent included in the marker material and/or coated on the marker material can vary. When two or more biological agents are included in and/or coated on the marker material, the amount of two or more biological agents can be the same or different.

In a further and/or alternative non-limiting aspect of the present invention, when one or more biological agents are included in the marker material, such biological agents can be released in a controlled manner from the marker material so the area in question to be treated is provided with the desired dosage of one or more biological agents over a sustained period of time. As can be appreciated, controlled release of one or more biological agents on the marker material is not always required and/or desirable. As such, one or more of the biological agents in the marker material can be uncontrollably released from the marker material during and/or after insertion of the medical device in the treatment area. It can also be appreciated that one or more biological agents in the marker material can be controllably released from the medical device and one or more biological agents in the marker material can be uncontrollably released from the medical device. As such, the marker material can be formulated such that 1) all the biological agent in the marker material is controllably released, 2) some of the biological agent in the marker material is controllably released and some of the biological agent is non-controllably released, or 3) none of the biological agent in the marker material is controllably released. The marker material can also be formulated such that the rate of release of the one or more biological agents in the marker material is the same or different. The adhesive material and/or the protective material of the marker material can be used at to least partially control the release of one or more biological agent from the marker material. One or more biological agents in the marker material can be separately coated when forming the marker material and/or be mixed with the adhesive material and/or protective material prior to applying the adhesive material and/or protective material to the medical device. The concentration of one or more biological agents, the type of protective material, the type of adhesive and/or the coating thickness of one or more biological agents can be used to control the release time, the release rate and/or the dosage amount of one or more biological agents; however, other or additional combinations can be used. As can also be appreciated, one or more biological agents can be deposited on the top surface of the marker material to provide an initial uncontrolled burst effect of the one or more biological agents when the medical device is inserted in a treatment area. The one or more biological agents can be coated on the medical device by a variety of mechanisms such as, but not limited to, spraying (e.g., atomizing spray techniques, air-brushing, spraying, etc.), dip coating, roll coating, sonication, brushing, plasma deposition, and/or depositing by vapor deposition. A masking technique can be used to limit the location of the biological agent on the medical device; however, this is not required. The thickness of each layer of biological agent is generally at least about 0.01 micron and is generally less than about 150 micron.

In still a further and/or alternative non-limiting aspect of the present invention, the marker material can have a color that is visible to the naked eye so as to enable a person to locate the marker material on a particular medical device. This information can then be used to facilitate in the determination of whether the medical device includes a marker material and/or confirm the location of the marker material on the medical so as to facilitate in guiding and/or positioning the medical device in a body passageway during a medical procedure. The marker material can be color coded to also or alternatively identify the device needed to locate the marker material on the medical device when the medical device is in a body passageway (e.g., x-ray machine, ultrasonic wave machine, etc.). This color coding can be in the form of a coloring agent that is coated on the surface of the marker material and/or contained in the marker material.

In yet a further and/or alternative non-limiting aspect of the present invention, the medical device that includes the marker material is used in the cardiology or neurology fields (e.g., balloon catheters, hypotubes, sheaths, stent catheters, PFO (patent foramen ovale) device, electrophysiology catheters, wires, guides, cutting devices, etc.). In one non-limiting embodiment, the marker material is included on a hypotube. In one particular design of this embodiment, the marker material forms at least a portion of a hypotube. In another and/or alternative particular design of this embodiment, the marker material is coated on one or more regions of the hypotube. In another non-limiting embodiment, the marker material is included on a stent. In one particular design of this embodiment, the marker material forms at least a portion of the stent. In another and/or alternative particular design of this embodiment, the marker material is coated on one or more regions of the stent. In still another non-limiting embodiment, the marker material is included on a guide catheter. In one particular design of this embodiment, the marker material forms at least a portion of the guide catheter. In another and/or alternative particular design of this embodiment, the marker material is coated on one or more regions of the guide catheter. In yet another non-limiting embodiment, the marker material is included on a guide wire. In one particular design of this embodiment, the marker material forms at least a portion of the guide wire. In another and/or alternative particular design of this embodiment, the marker material is coated on one or more regions of the guide wire. In still yet another non-limiting embodiment, the marker material is included on a balloon. In one particular design of this embodiment, the marker material forms at least a portion of the balloon. In another and/or alternative particular design of this embodiment, the marker material is coated on one or more regions of the balloon. As can be appreciated, the marker material can be coated on and/or form a portion of other medical devices. The one or more layers of the marker material can be coated on the medical device by one or more techniques such as, but not limited to, spraying (e.g., atomizing spray techniques, air-brushing, spraying, etc.), dip coating, roll coating, sonication, brushing, plasma deposition, and/or depositing by vapor deposition, etc. A masking technique can be used to limit the location of the marker material on the medical device; however, this is not required. The coating thickness of each layer of the marker material may or may not have a uniform thickness. The location of the marker material can be on one or multiple locations on the medical device. The size of the one or more regions that include the marker material can be the same or different. The marker material can be spaced at defined distances from one another so as to form ruler like markings on the medical device to facilitate in the positioning of the medical device in a body passageway.

It is one non-limiting object of the invention to provide a marker material that is flexible when used on a medical device.

It is another and/or alternative non-limiting object of the invention to provide a marker material that results in little or no added bulk to a medical device.

It is still another and/or alternative non-limiting object of the invention to provide a marker material that includes a protective material coating.

It is yet another and/or alternative non-limiting object of the invention to provide a marker material that includes an adhesive material coating.

It is still yet another and/or alternative non-limiting object of the invention to provide a marker material that includes a metal powder.

It is a further and/or alternative non-limiting object of the invention to provide a marker material that includes a biological agent.

These and other objects and advantages will become apparent from the following description taken together with the accompanying drawings.

### BRIEF DESCRIPTION OF THE DRAWINGS

Reference may now be made to the drawings, which illustrate an embodiment that the invention may take in physical form and in certain parts and arrangements of parts wherein;

FIG. 1 is a cross-sectional view of a prior art medical device in a blood vessel, wherein the prior art medical device includes a hypotube having two marker bands, a balloon connected to the end section of the hypotube and a stent crimped to the balloon, and which the prior art medical device cannot be further moved along a guide wire due to narrowing in the blood vessel;

FIG. 2 is a cross-sectional view of a prior art medical device in a blood vessel, wherein the prior art medical device includes a hypotube having two marker bands and a balloon connected to the end section of the hypotube, and which the prior art medical device cannot be further moved along a guide wire due to narrowing in the blood vessel;

FIG. 3 is a cross-sectional view of a medical device in a blood vessel, wherein the medical device includes a hypotube having two flexible marker bands of the present invention, a balloon connected to the end section of the hypotube and a stent crimped to the balloon, and which the medical device is able to be further moved along a guide wire to a diseased area in the blood vessel;

FIG. 4 is a cross-sectional view of the medical device of FIG. 3 that has been expanded in a blood vessel;

FIG. 5 is a cross-sectional view of a medical device in a blood vessel, wherein the medical device includes a hypotube having two flexible marker bands of the present invention and a balloon connected to the end section of the hypotube, and which the medical device is able to be further moved along a guide wire to a diseased area in the blood vessel;

FIG. 6 is a cross-sectional view of the medical device of FIG. 5 that has been expanded in a blood vessel;

FIG. 7 is a cross-sectional view of the medical device in a blood vessel, wherein the medical device is a stent in an expanded state and includes a flexible marker material of the present invention at each end of the stent and a protective coating over the marker material;

FIG. 8 is a cross-sectional view along lines 8-8 of FIG. 7;

FIG. 9, is a similar cross-sectional view as FIG. 8 except that the marker material is shown to be coated over a larger region of the stent; and,

FIG. 10 is a cross-sectional view of another arrangement of the marker material coated on a portion of the stent.

### DETAILED DESCRIPTION OF THE INVENTION

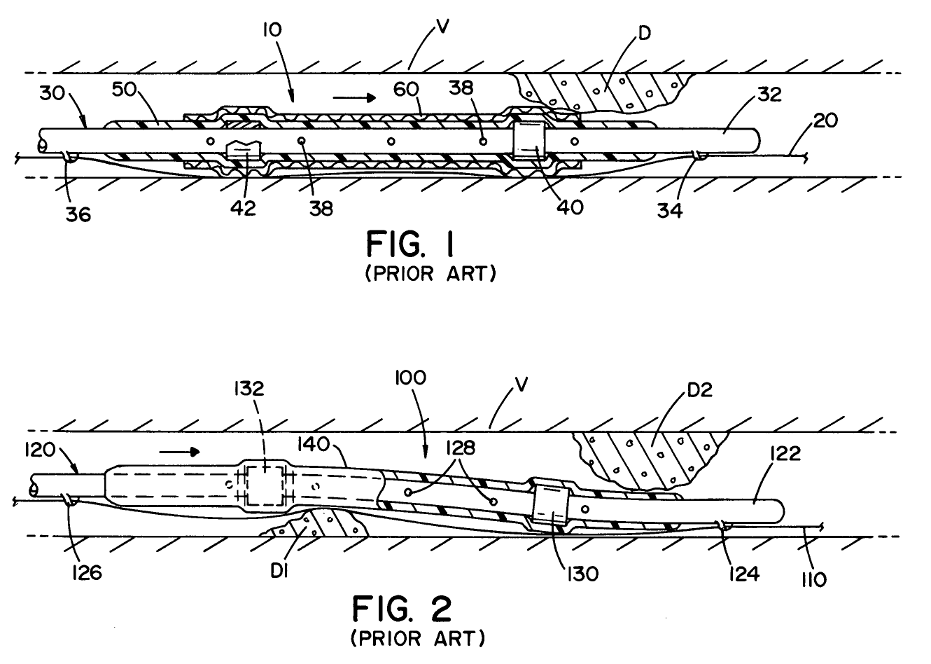

Referring now to the drawings wherein the showings are for the purpose of illustrating the preferred embodiments only and not for the purpose of limiting the same, FIGS. 1 and 2 illustrate prior art medical devices that incorporate the use of prior art, rigid and bulky metal marker bands. Referring now to FIG. 1, there is shown a medical device 10 positioned in a blood vessel V. The inner region of blood vessel V includes a deposit D which forms a diseased area in the blood vessel. Medical device 10 is shown as being guided along guide wire 20 to the diseased area of the blood vessel. The medical device 10 includes a hypotube 30 having an end section 32 and two connectors 34, 36 on the end section. The two connectors allow the guide wire 20 to be threaded through the connectors so that the hypotube can be guided along the guide wire to the diseased area. As can be appreciated, the guide wire can engage the hypotube in other and/or additional ways. The end section includes a plurality of openings 38 that are used to enable fluid (e.g., gas and/or liquid) that is flowing through the internal channel of a the hypotube to inflate balloon 50 that is secure to the end section of the hypotube. As can be appreciated, the hypotube can include other and/or additional structures to inflate the balloon. The balloon can be connected to the hypotube in a variety of ways (e.g., adhesive, welding, melting, formed as part of the hypotube, etc.). The end section 32 of the hypotube also includes two metal marker bands 40, 42. These marker bands are typically radiopaque. The metal marker bands are made of a rigid metal that does not easily bend. The metal marker bands also have a thickness that results in an increased thickness of the medical device in the area of the marker bands. A stent 60 is also illustrated as being crimped to balloon 50 so as to secure the stent to the balloon. As can be appreciated, the stent can be secured to the balloon in other and/or additional ways. As stated above, the two marker bands on the hypotube add thickness to the medical device. As shown in FIG. 1, the added thickness of the marker bands prevent the medical device from being properly positioned in the blood vessel to treat the diseased area. As a result, the diseased area is not treated or a different medical procedure has to be used to treat the diseased area.

Referred now to FIG. 2, there is shown another medical device 100 positioned in a blood vessel V. The inner region of blood vessel V includes two deposits D1 and D2 which form diseased areas in the blood vessel. D2 is the larger diseased area and is to be treated by the medical device. Medical device 100 is shown as being guided along guide wire 110 to diseased area D2 in blood vessel V. The medical device 100 includes a hypotube 120 having an end section 122 and two connectors 124, 126 on the end section. The two connectors allow the guide wire 110 to be threaded through the connectors so that the hypotube can be guided along the guide wire to the diseased area. As can be appreciated, the guide wire can engage the hypotube in other and/or additional ways. The end section includes a plurality of openings 128 that are used to enable fluid (e.g., gas and/or liquid) that is flowing through the internal channel of a the hypotube to inflate balloon 140 that is secure to the end section of the hypotube. As can be appreciated, the hypotube can include other and/or additional structures to inflate the balloon. The balloon can be connected to the hypotube in a variety of ways (e.g., adhesive, welding, melting, formed as part of the hypotube, etc.). The end section 122 of the hypotube also includes two metal marker bands 130, 132. These marker bands are typically radiopaque. The metal marker bands are made of a rigid metal that does not easily bend. The metal marker bands also have a thickness than results in an increased thickness of the medical device in the area of the marker bands. The two marker bands 130, 132 on the hypotube add thickness to the medical device. As shown in FIG. 2, the added thickness of the marker band prevents the medical device from being properly positioned in the blood vessel to treat the diseased area D2. As a result, the diseased area is not treated or a different medical procedure has to be used to treat the diseased area.

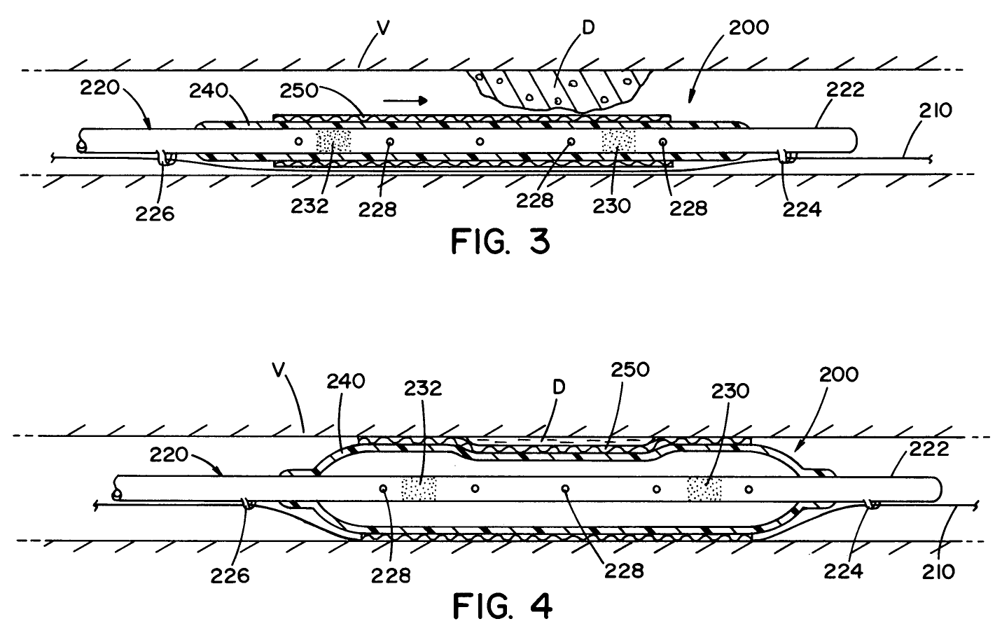

Referring now to FIGS. 3 and 4, there is illustrated a medical device 200 in accordance with the present invention. Medical device 200 is shown to be positioned in a blood vessel V. The inner region of blood vessel V includes a deposit D which forms a diseased area in the blood vessel. Medical device 200 is shown as being guided along guide wire 210 to the diseased area of the blood vessel. The medical device 200 includes a hypotube 220 having an end section 222 and two connectors 224, 226 on the end section. The two connectors allow the guide wire 210 to be threaded through the connectors so that the hypotube can be guided along the guide wire to the diseased area. As can be appreciated, the guide wire can engage the hypotube in other and/or additional ways. The end section includes a plurality of openings 228 that are used to enable fluid (e.g., gas and/or liquid) that is flowing through the internal channel of a the hypotube to inflate balloon 240 that is secure to the end section of the hypotube. As can be appreciated, the hypotube can include other and/or additional structures to inflate the balloon. The balloon can be connected to the hypotube in a variety of ways (e.g., adhesive, welding, melting, formed as part of the hypotube, etc.). The end section 222 of the hypotube also includes two bands of flexible marker material 230, 232. As can be appreciated, the hypotube can include less bands or more bands of flexible marker material. These bands of flexible marker material are typically radiopaque; however, this is not required. The flexible marker bands can be a part of the hypotube itself and/or be coated on the outer surface of the hypotube. The bands of the flexible marker material can extend completely or partially about the circumference of the hypotube. A stent 250 is illustrated as being crimped to balloon 240 to secure the stent to the balloon. As can be appreciated, the stent can be secured to the balloon in other and/or additional ways. Although not shown, the stent can also or alternatively include the flexible marker material. As stated above, the two marker bands on the hypotube do not add any significant thickness to the medical device. As shown in FIG. 4, the medical device is able to be properly positioned in the blood vessel to treat the diseased area. This is a significant improvement over the medical device 10 of FIG. 1 which could not pass by deposit D1 due to the thickness of standard metal marker bands. The bands of flexible marker material 230, 232 on hypotube 220 are used to properly position the balloon and the stent in the diseased area of the blood vessel. Once the medical device is properly positioned in the blood vessel, balloon 240 is expanded which in turn expands stent 250 as illustrated in FIG. 4. The expansion of the balloon and the stent compresses deposit D and opens the narrowed region of the blood vessel formerly caused by deposit D. After the stent is expanded, the balloon is deflated and the balloon, hypotube and guide wire are removed from the blood vessel.

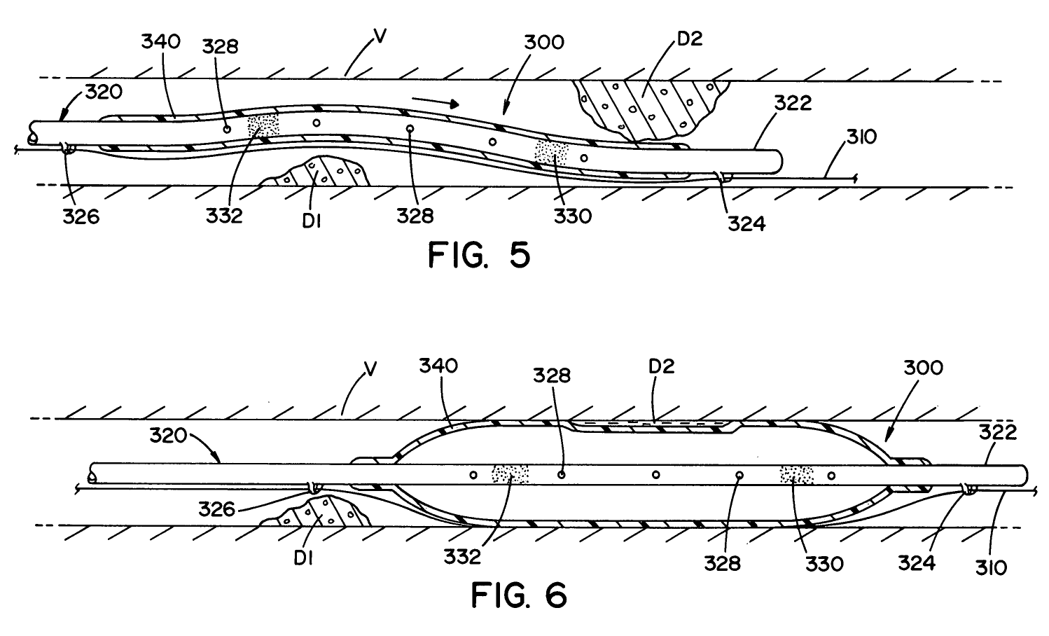

Referring now to FIGS. 5 and 6, there is illustrated a medical device 300 in accordance with the present invention. Medical device 300 is shown to be positioned in a blood vessel V. The inner region of blood vessel V includes two deposits D1 and D2 which form a diseased area in the blood vessel. D2 is the larger diseased area and is to be treated by the medical device. Medical device 300 is shown as being guided along guide wire 310 to the diseased area of the blood vessel. The medical device 300 includes a hypotube 320 having an end section 322 and two connectors 324, 326 on the end section. The two connectors allow the guide wire 310 to be threaded through the connectors so that the hypotube can be guided along the guide wire to the diseased area. As can be appreciated, the guide wire can engage the hypotube in other and/or additional ways. The end section includes a plurality of openings 328 that are used to enable fluid (e.g., gas and/or liquid) that is flowing through the internal channel of the hypotube to inflate balloon 340 that is secure to the end section of the hypotube. As can be appreciated, the hypotube can include other and/or additional structures to inflate the balloon. The balloon can be connected to the hypotube in a variety of ways (e.g., adhesive, welding, melting, formed as part of the hypotube, etc.). The end section 322 of the hypotube also includes two bands of flexible marker material 330, 332. As can be appreciated, the hypotube can include less bands or more bands of flexible marker material. These bands of flexible marker material are typically radiopaque; however, this is not required. The bands of flexible marker material can be a part of the hypotube itself and/or be coated on the outer surface of the hypotube. The bands of the flexible marker material can extend completely or partially about the circumference of the hypotube. As can be appreciated, the flexible marker material can also or alternatively form a part of and/or be placed on balloon 340. As shown in FIG. 6, the medical device is able to serpentine about deposits D1 and D2 due to the flexibility of the bands of flexible marker material, and is able to be properly positioned in the blood vessel to treat the diseased area. This is a significant improvement over the medical device 100 of FIG. 2 which was less flexible due to the metal marker bands and also which could not pass by deposit D2 due to the thickness of standard metal marker bands. The bands of flexible marker material 330, 332 on hypotube 320 are used to properly position the balloon in the diseased area of the blood vessel. Once the medical device is properly positioned in the blood vessel, balloon 240 is expanded as illustrated in FIG. 6. The expansion of the balloon compresses deposit D2 and opens the narrowed region of the blood vessel formerly caused by deposit D2. After the balloon is expanded, the balloon is deflated and the balloon, hypotube and guide wire are removed from the blood vessel.

The one or more bands of flexible marker material are formed by at least three components, namely an adhesive material, a metal powder and a protective material. These three components can be applied as distinct layers on the hypotube or two or more of these components can be mixed together prior to being applied to and/or forming at least a portion of the hypotube. Typically the marker material only includes one layer of each material; however, this is not required. When the flexible marker material is coated on the hypotube, the final average coating thickness of the marker material is generally less than about 500 microns and typically about 20-100 microns. When the marker material is coated on the hypotube, the adhesive material is typically applied first; however, it can be appreciated that the metal powder layer can be at least partially applied prior to or during the application of the adhesive material to the hypotube. The layer of adhesive material is at least partially designed to retain the layer of particles of metal powder on the hypotube. A variety of adhesives can be used. One non-limiting adhesive material is a silicone based adhesive such as, but not limited to, a silastic silicone rubber offered by Dow Coming. As can be appreciated, other or additional types of adhesive material can be used. The thickness of the layer of adhesive material on the hypotube is typically less than about 40 microns and typically about 2-10 microns; however, other thicknesses can be used. As can be appreciated, if the adhesive material is mixed with metal powder and/or protective material, the thickness of the adhesive layer will typically be greater than 10 microns. When the adhesive material is applied to the hypotube, the adhesive material can be first diluted to reduce the viscosity of the adhesive material so that the desired thin coating layer of the adhesive material can be obtained. When this coating of adhesive material is applied to the hypotube, the adhesive material is typically applied to the hypotube by a spraying technique or bay vapor deposition; however, this is not required. After the layer of adhesive material is applied to the hypotube, a layer of metal powder particles 440 is applied to the layer of adhesive material. Typically the metal powder is applied to the layer of adhesive material prior to the adhesive material fully setting, thus allowing the metal powder to at least partially adhere to the adhesive material. As can be appreciated, all or a portion of the metal powder can be applied to the hypotube prior to applying the adhesive material to the hypotube or after the adhesive material is applied to the hypotube. As also can be appreciated, all or a portion of the metal powder can be combined with the adhesive material prior to the adhesive material being applied to the hypotube. When the layer of metal powder is applied to the layer of adhesive material, the metal powder can be applied 1) in a substantially pure powder form, 2) applied as a mixture of metal powder and adhesive material, 3) applied as a mixture of metal powder and protective material, or 4) applied as a mixture of metal powder, adhesive material and protective material. The metal powder typically includes a majority of bismuth, molybdenum, rhenium, tungsten and/or an alloy of one or more of such metals. These metals are radiopaque and function primarily to increase the visibility of the hypotube during a medical procedure. The average particle size of about 99.9% of the metal powder is generally less than about 75 microns and typically about 5-50 microns; however, other particles sizes can be used. The metal powder generally constitutes a majority weight percent of the marker material and typically at least about 75 weight percent of the marker material. The metal powder, when applied in its substantially pure form (i.e., not mixed with adhesive material and/or protective material), is typically applied by controllably spraying or sprinkling the metal powder on the layer of adhesive material; however, other techniques can be used. The coating thickness of the metal powder, when applied in its substantially pure form, is generally less than about 150 microns and typically about 5-100 microns; however, other thicknesses can be used. Coated on the surface of the layer of metal powder is a layer of protective material 430. The protective material typically includes one or more polymer materials that are formulated to 1) form a barrier between the metal powder and/or adhesive material on the hypotube and the body passageway and/or fluids in the body passageway, 2) facilitate in retaining and/or adhering the metal powder particles and/or adhesive on the hypotube, 3) shield and/or protect the metal powder particle and/or adhesive material from damage a) during the insertion of the hypotube in the body passageway, b) when inserting a balloon and/or other type of device on the hypotube, and/or c) during the handling of the hypotube, and/or 4) providing a desired surface profile on the surface of the hypotube. The protective material is typically a biocaptable material and a biostable material; however, this is not required. The one or more polymers that can be used to at least partially form the protective material include, but are not limited to, parylene, PLGA, POE, PGA, PLLA, PAA, PEG, chitosan and/or derivatives of one or more of these polymers. Typically the protective material includes or is fully formed from parylene and/or a derivative thereof (e.g., parylene c, etc.); however, this is not required. The coating thickness of the protective material, when applied in its substantially pure form (i.e. protective material without the inclusion of metal powder and/or adhesive material), is generally less than about 20 microns and typically about 0.3-4 microns; however, other thicknesses can be used. The protective material is typically coated on the hypotube by a spraying technique or a vapor deposition technique; however, this is not required. When the protective material is coated on the hypotube, the coating area of the protective material is selected to cover over 90 percent of the adhesive material and/or metal powder that was previously coated on the hypotube, and typically about 95-100 percent of the adhesive material and/or metal powder that was previously coated on the hypotube. The marker material 420 can include one or more biological agents that are coated on the surface of the marker material and/or contained in the marker material; however, this is not required. If one or more biological agents are used, one or more of these biological agents can be controllably released from the marker material; however, this is not required. The marker material can include a coloring agent that is coated on the surface of the marker material and/or contained in the marker material; however, this is not required.

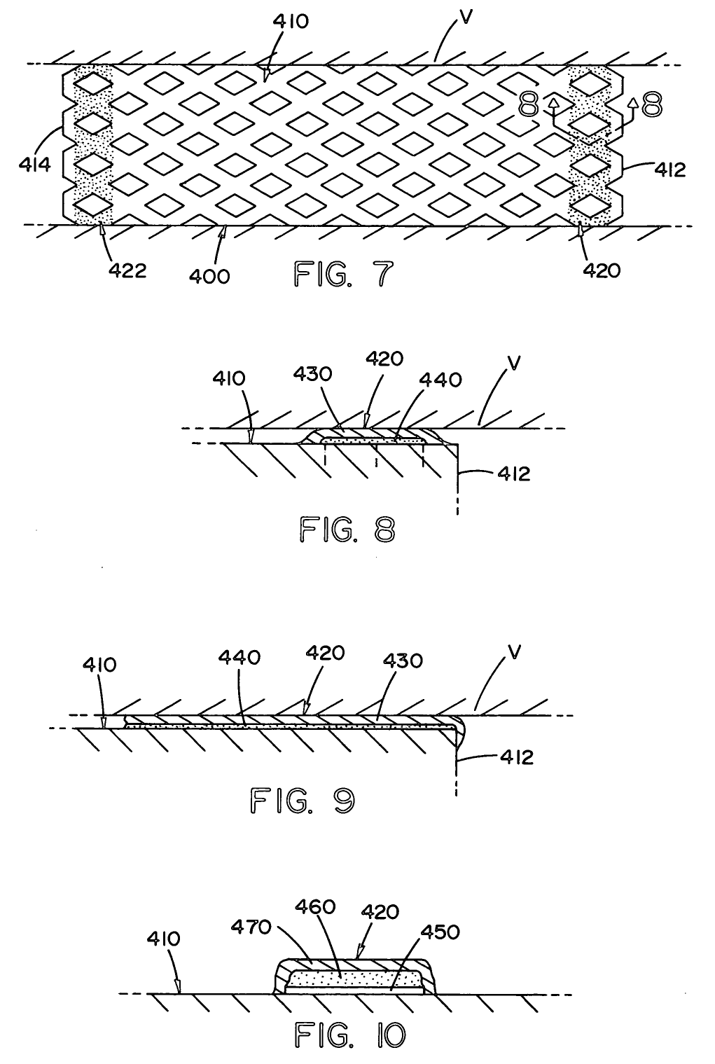

Referring now to FIGS. 7-10, there is illustrated a stent 400 that includes a flexible marker material 420, 422 positioned on one or more portions of a stent. The stent includes one or more body members 410, wherein each body member includes first and second ends 412, 414 and a wall surface disposed between the first and second ends. Each body member has a first cross-sectional area which permits delivery of the stent into blood vessel V, and a second, expanded cross-sectional area. The expansion of the stent can be accomplished in a variety of manners. Typically, the stent is expanded to its second cross-sectional area by a radially, outwardly extending force applied at least partially from the interior region of the stent (e.g., by use of a balloon, etc.). Alternatively, or additionally, the stent can include heat sensitive materials (e.g., shape memory materials, etc.) that expand upon exposure to heat. The second cross-sectional area of the stent can be fixed or variable. When the second cross-sectional area is variable, the second cross-sectional area is typically dependent upon the amount of radially outward force applied to the stent. The stent can be designed such that the stent expands while retaining the original length of the stent; however, this is not required. The stent can have a first cross-sectional shape that is generally circular so as to form a substantially tubular stent; however, the stent can have other cross-sectional shapes. When the stent includes two or more body members, the two or more body members can be connected together by at least one connector member, not shown. The stent can include rounded, smooth and/or blunt surfaces to minimize and/or prevent damage to a blood vessel as the stent is inserted into a blood vessel and/or expanded in a blood vessel; however, this is not required. The stent can be formed of a variety of materials (e.g., metal, polymer, etc.). One or more portions of the stent can be biostable or biodegradable.

The flexible marker material 420, 422 is typically radiopaque; however, this is not required. One or more bands of flexible marker material can form a part of the stent itself and/or be coated on the outer surface of the stent. When the marker material forms a portion of the stent, the region of the stent that includes the flexible marker material is typically the same thickness as other regions of the stent; however, this is not required. When the flexible marker material is coated on the surface of the stent, the coating thickness is generally less than about 500 microns and typically about 20-100 microns so as to not add any significant thickness to the stent in the areas that include the flexible marker material. The marker material is illustrated as being positioned at the ends of the stent; however, it can be appreciated that the marker material can be positioned in other or additional regions of the stent.

Referring now to FIG. 8, the marker material 420 is shown to be coated on specific regions of the surface of the stent. A protective material coating 430 is illustrated as being coated over the complete surface of the layer of metal powder particles 440. As can be appreciated, the layer of metal powder particles can include an adhesive material; however, this is not required. The protective material is shown to substantially fully encapsulate the metal powder particles between the surface of the stent and the protective material. In this non-limiting arrangement, the protective material facilitates in 1) forming a barrier between the metal powder and/or adhesive material on the stent and the body passageway and/or fluids in the body passageway, 2) retaining and/or adhering the metal powder particles and/or adhesive on the stent, 3) shield and/or protect the metal powder particle and/or adhesive material from damage a) during the insertion of the stent in the body passageway, b) when inserting the stent on a balloon and/or other type of delivery device, and/or c) during the handling of the stent, and/or 4) providing a desired surface profile on the surface of the stent.

Referring now to FIG. 9, there is illustrated a section of a stent similar to the section illustrated in FIG. 8. As shown in FIG. 9, the coating of marker material coating covers a larger region of the surface of the stent. As can be appreciated, up to the complete stent can be coated with the flexible marker material.

Referring now to FIG. 10, there is illustrated a section of a stent that includes a marker material 420 formed from three layers of material. These three components can be applied as distinct layers on the stent or two or more of these components can be mixed together prior to being applied to and/or forming at least a portion of the hypotube. The final average coating thickness of the marker material is generally less than about 500 microns and typically about 20-100 microns. As illustrated in FIG. 10, the marker material includes one layer of each material. The first layer of the flexible marker material is formed by an adhesive material 450. The layer of adhesive material is at least partially designed to retain the layer of particles of metal powder 460 on the stent. A variety of adhesives can be used. One non-limiting adhesive material is a silicone based adhesive such as, but not limited to, a silastic silicone rubber offered by Dow Coming. As can be appreciated, other or additional types of adhesive material can be used. The thickness of the layer of adhesive material on the stent is typically less than about 40 microns and typically about 2-10 microns; however, other thicknesses can be used. As can be appreciated, if the adhesive material is mixed with metal powder and/or protective material, the thickness of the adhesive layer will typically be greater than 10 microns. When the adhesive material is applied to the stent, the adhesive material can be first diluted to reduce the viscosity of the adhesive material so that the desired thin coating layer of the adhesive material can be obtained. When this coating of adhesive material is applied to the stent, the adhesive material is typically applied to the stent by a spraying technique or bay vapor deposition; however, his is not required. After the later of adhesive material is applied to the stent, a layer of metal powder particles 460 is applied to the layer of adhesive material. Typically the metal powder is applied to the layer of adhesive material prior to the adhesive material fully setting, thus allowing the metal powder to at least partially adhere to the adhesive material. As can be appreciated, all or a portion of the metal powder can be applied to the stent prior to applying the adhesive material to the stent or after the adhesive material is applied to the stent. As also can be appreciated, all or a portion of the metal powder can be combined with the adhesive material prior to the adhesive material being applied to the stent. When the layer of metal powder is applied to the layer of adhesive material, the metal powder can be applied 1) in a substantially pure powder form, 2) applied as a mixture of metal powder and adhesive material, 3) applied as a mixture of metal powder and protective material, or 4) applied as a mixture of metal powder, adhesive material and protective material. FIG. 10 illustrates the layer of metal powder in a substantially pure form. The metal powder typically includes a majority of bismuth, molybdenum, rhenium, tungsten and/or an alloy of one or more of such metals. These metals are radiopaque and function primarily to increase the visibility of the stent during a medical procedure. The average particle size of about 99.9% of the metal powder is generally less than about 75 microns and typically about 5-50 microns; however, other particles sizes can be used. The metal powder generally constitutes a majority weight percent of the marker material and typically at least about 75 weight percent of the marker material. The metal powder, when applied in its substantially pure form (i.e., not mixed with adhesive material and/or protective material), is typically applied by controllably spraying or sprinkling the metal powder on the layer of adhesive material; however, other techniques can be used. The coating thickness of the metal powder, when applied in its substantially pure form, is generally less than about 150 microns and typically about 5-100 microns; however, other thicknesses can be used. Coated on the surface of the layer of metal powder is a layer of protective material 470. The protective material typically includes one or more polymer materials that are formulated to 1) form a barrier between the metal powder and/or adhesive material on the stent and the body passageway and/or fluids in the body passageway, 2) retain and/or adhere the metal powder particles and/or adhesive on the stent, 3) shield and/or protect the metal powder particle and/or adhesive material from damage a) during the insertion of the stent in the body passageway, b) when inserting the stent on a balloon and/or other type of delivery device, and/or c) during the handling of the stent, and/or 4) provide a desired surface profile on the surface of the stent. The protective material is typically a biocaptable material and a biostable material; however, this is not required. The one or more polymers that can be used to at least partially form the protective material include, but are not limited to, parylene, PLGA, POE, PGA, PLLA, PAA, PEG, chitosan and/or derivatives of one or more of these polymers. Typically the protective material includes or is fully formed from parylene and/or a derivative thereof (e.g., parylene c, etc.); however, this is not required. The coating thickness of the protective material, when applied in its substantially pure form (i.e. protective material without the inclusion of metal powder and/or adhesive material), is generally less than about 20 microns and typically about 0.3-4 microns; however, other thicknesses can be used. The protective material is typically coated on the stent by a spraying technique or a vapor deposition technique; however, this is not required. When the protective material is coated on the stent, the coating area of the protective material is selected to cover over 90 percent of the adhesive material and/or metal powder that was previously coated on the stent, and typically about 95-100 percent of the adhesive material and/or metal powder that was previously coated on the stent. The marker material 420 can include one or more biological agents that are coated on the surface of the marker material and/or contained in the marker material; however, this is not required. If one or more biological agents are used, one or more of these biological agents can be controllably released from the marker material; however, this is not required. The marker material can include a coloring agent that is coated on the surface of the marker material and/or contained in the marker material; however, this is not required.

The embodiments of the invention set forth in FIGS. 1-10 all relate to the use of the marker material on certain types of medical devices for use in various types of vascular procedures. It will be appreciated that the marker material can be used on other types of medical devices used in vascular procedures. It will also be appreciated that the marker material can be used on a medical device for insertion in body passageways other than vascular passageways or used on medical devices that are inserted in other regions of the body (e.g., prosthetic device, etc.).

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall therebetween.

We claim:

1. A medical device that includes a body portion having at least one marker material, said marker material visible to at least one form of electromagnetic wave, sound wave, magnetic wave or combinations thereof, said marker material formed of a flexible and non-rigid material, said marker material including adhesive material, metal powder and protective material.

2. The medical device as defined in claim 1, wherein said medical device is at least partially coated with said marker material.

3. The medical device as defined in claim 1, wherein said adhesive material includes a silicone adhesive, a silicone based adhesive, a polyurethane adhesive, a polyurethane based adhesive, an acrylic adhesive, an acrylic based adhesive, or combinations thereof.

4. The medical device as defined in claim 1, where in said adhesive layer has an average thickness of about 2-50 microns.

5. The medical device as defined in claim 3, where in said adhesive layer has an average thickness of about 2-50 microns.

6. The medical device as defined in claim 1, wherein a majority weight percent of said metal powder includes bismuth, molybdenum, rhenium, tungsten, or combinations thereof.

7. The medical device as defined in claim 5, wherein a majority weight percent of said metal powder includes bismuth, molybdenum, rhenium, tungsten, or combinations thereof.

8. The medical device as defined in claim 1, wherein at least about 95 percent of said metal powder has an average particle size of about 5-150 microns.

9. The medical device as defined in claim 7, wherein at least about 95 percent of said metal powder has an average particle size of about 5-150 microns.

10. The medical device as defined in claim 1, wherein a majority of said metal powder is in the form of a metal layer that is positioned on a layer of said adhesive material.

11. The medical device as defined in claim 1, wherein a majority of said metal powder is radiopaque.

12. The medical device as defined in claim 1, wherein said protective material covers at least about 95 percent of said metal powder.

13. The medical device as defined in claim 1, wherein said protective material has a coating thickness of about 0.1-10 microns.

14. The medical device as defined in claim 10, wherein said protective material has a coating thickness of about 0.1-10 microns.

15. The medical device as defined in claim 1, wherein said protective material includes at least one polymer, said at least one polymer including parylene, parylene derivatives or combinations thereof

16. The medical device as defined in claim 14, wherein said protective material includes at least one polymer, said at least one polymer including parylene, parylene derivatives or combinations thereof.

17. The medical device as defined in claim 1, wherein said medical device is a device designed to be positioned in a body passageway, said device including a hypotube, a balloon, a sheath, a guide catheter, an electrophysiology catheter, a guide wire, a stent, a graft, a PFO device, or combinations thereof.

18. The medical device as defined in claim 1, wherein said marker material includes at least one biological agent.

19. The medical device as defined in claim 1, wherein said marker material includes color agent.

20. A method of forming a flexible marker on a medical device including:
a) providing a medical device having an outer surface;
b) applying a layer of an adhesive material to at least a portion of said outer surface of said medical device;
c) applying a layer of metal material to at least a portion of said adhesive material, said metal layer including metal powder, metal salt or combinations thereof, said metal layer visible to at least one form of electromagnetic wave, sound wave, magnetic wave or combinations thereof; and,
d) applying a protective material over said layer of said adhesive material and said layer of metal material so as to cover at least about 90 percent of said two layers.

21. The method as defined in claim 20, wherein said adhesive material includes a silicone adhesive, a silicone based adhesive, a polyurethane adhesive, a polyurethane based adhesive, an acrylic adhesive, an acrylic based adhesive, or combinations thereof.

22. The method as defined in claim 20, wherein said adhesive layer has an average thickness of about 2-50 microns.

23. The method as defined in claim 20, wherein a majority weight percent of said metal material is a metal powder that includes bismuth powder, molybdenum powder, rhenium powder, tungsten powder, or combinations thereof.

24. The method as defined in claim 20, wherein at least about 95 percent of said metal material has an average particle size of about 5-150 microns.

25. The method as defined in claim 20, wherein a majority of said metal material is radiopaque.

26. The method as defined in claim 20, wherein said protective material has a coating thickness of about 0.1-10 microns.

27. The method as defined in claim 20, wherein said protective material includes at least one polymer, said at least one polymer including parylene, parylene derivatives or combinations thereof.

28. The method as defined in claim 20, wherein said medical device is a device designed to be positioned in a body passageway, said device including a hypotube, a balloon, a sheath, a guide catheter, an electrophysiology catheter, a guide wire, a stent, a graft, a PFO device, or combinations thereof.

29. The method as defined in claim 20, including the step of applying at least one layer of biological agent to said adhesive material, said metal material or combinations thereof prior to applying said layer of protective material.

30. The method as defined in claim 20, including the step of applying at least one layer of biological agent to said layer of protective material.

31. The method as defined in claim 20, including the step of including a color agent in said flexible marker.

32. The method as defined in claim 20, including the use of a mask to at least partially control the location of said adhesive, said metal material, said protective material, or combinations thereof being applied to said medical device.

33. A method of forming a flexible marker on a medical device including:
a) providing a medical device having an outer surface; and,
b) applying a layer of an adhesive material, a layer of a radiopaque material and a layer of protective material on said outer surface of said medical device.

34. The method as defined in claim 33, including the step of applying said layer of radiopaque material to at least a portion of said adhesive material, said radiopaque material including metal powder, metal salt or combinations thereof.

35. The method as defined in claim 33, including the step of applying said layer of protective material over said layer of said adhesive material, said layer of radiopaque material, or combinations thereof.

36. The method as defined in claim 35, wherein said layer of protective material covering at least about 90 percent of said layer of radiopaque material.

38. The method as defined in claim 33, where in said adhesive layer has an average thickness of about 2-50 microns.

39. The method as defined in claim 33, wherein at least about 95 percent of said radiopaque material has an average particle size of about 5-150 microns.

40. The method as defined in claim 33, wherein said protective material has a coating thickness of about 0.1-10 microns.

41. The method as defined in claim 33, wherein said adhesive material is at least partially applied to said medical device by spraying, air brushing, painting, dip coating, vapor deposition, or combinations thereof.

42. The method as defined in claim 33, wherein said radiopaque material is at least partially applied to said medical device by spraying, air brushing, painting, dip coating or combinations thereof.

43. The method as defined in claim 42, wherein said radiopaque material is applied to said medical device in a substantially even, fluent coating.

44. The method as defined in claim 33, wherein said radiopaque material is at least partially mixed with said adhesive material prior to being applied to said medical device.

45. The method as defined in claim 33, wherein said protective material is at least partially applied to said medical device by spraying, air brushing, painting, dip coating, vapor deposition or combinations thereof.

46. The method as defined in claim 33, including the step of applying at least one layer of biological agent to said adhesive material, said radiopaque material or combinations thereof prior to applying said layer of protective material.

47. The method as defined in claim 33, including the step of applying at least one layer of biological agent to said layer of protective material.

48. The method as defined in claim 33, including the step of including a color agent in said flexible marker.

49. The method as defined in claim 33, including the use of a mask to at least partially control the location of said adhesive, said radiopaque material, said protective material, or combinations thereof being applied to said medical device.
